(19) United States
(12) Patent Application Publication
(10) Pub. No.: US 2006/0161213 A1
(43) Pub. Date: Jul. 20, 2006

(54) Method of graphical display of link status and fail-safe mechanism for safety during real-time medical device therapy

(76) Inventor: Neilesh S. Patel, Los Altos, CA (US)

Correspondence Address: MEDTRONIC, INC., 710 MEDTRONIC PARK, MINNEAPOLIS, MN, 55432-9924, US

(21) Appl. No.: 11/036,243

(22) Filed: Jan. 14, 2005

(51) Int. Cl. A61N 1/372

(52) U.S. Cl. 607/30; 607/60

(57) ABSTRACT

A remote medical device programming system and method are provided for monitoring a communication signal during a remote programming procedure and generating a communication status signal for presentation on a display. The communication signal monitoring may include monitoring a communication link bandwidth on a communication network linking a remote programmer to a central programmer. The communication signal monitoring may also include monitoring a telemetry signal strength between the remote programmer and the medical device being programmed. The system and method further provide a fail-safe response to a communication status signal that does not meet predetermined safety criteria.

### FIELD OF THE INVENTION

The invention relates to implantable medical devices, and more particularly, to techniques for remote programming of implantable medical devices.

### BACKGROUND OF THE INVENTION

A variety of programmable implantable medical devices (IMD) are available which typically utilize radio frequency (RF) telemetry for communicating with an external programmer. Such devices include cardiac pacemakers and defibrillators, drug pumps, neuromuscular stimulators, hemodynamic monitors and other physiological monitors. Programming commands or data are transmitted between an implanted RF telemetry antenna and associated circuitry and an external RF telemetry antenna associated with the external programmer. In the past, the external RF telemetry antenna has been contained in a programmer RF head so that it can be located close to the patient's skin overlying the IMD. Programmer RF heads are well known in the art. See for example U.S. Pat. No. 4,550,370 issued to Baker, incorporated herein by reference in its entirety. Such telemetry systems may be referred to as “near-field” systems because they require the external antenna to be positioned in close proximity to the implanted antenna in order to establish and maintain telemetry communication.

Long range or far-field telemetry systems have been introduced wherein the external telemetry antenna may be located a distance away from the patient, for example a few meters or so from the patient, during telemetry communication. The external RF telemetry antenna can be located on the case of the programmer or otherwise integrated with the programmer or an associated stand or cart. Such long-range telemetry systems are generally preferable over systems requiring an RF head because it frees the hands of the clinician for performing other tasks during an interrogation or programming session. Moreover, the patient may be active, e.g., performing treadmill or other exercise or stress testing, during a telemetry transmission. Telemetry systems that do not require the use of a programmer RF head are generally disclosed in U.S. Pat. No. 6,240,317 issued to Villaseca et al., U.S. Pat. No. 6,169,925 Villaseca et al., and U.S. Pat. No. 6,482,154 Haubrich et al., all of which patents are incorporated herein by reference in their entirety.

A number of proposals have been made to enable remote programming and monitoring of an IMD from a centralized patient management center. Such systems generally rely on a home monitoring device having bidirectional communication with the implanted device for retrieving or transferring data from/to the IMD. The home monitor is coupled to a data communication network to enable transfer of data to/from a centralized database or medical support network. The home monitor functions as a communication conduit between the central patient management center and the IMD.

In order to ensure the safety of the patient, data transferred from a central patient management center to the home monitor must be safely and securely transferred over the communication network without data loss or corruption. Programming changes that affect the operation of the implantable medical device may affect the health and safety of the patient. Safety issues during remote programming procedures are particularly important because the patient may not be in the presence of medical personnel or emergency equipment. Communication systems which promote safe remote programming of medical devices are therefore needed. By providing safe, secure remote programming of medical devices, patient management procedures may be enhanced by providing patients with timely responses to changes in medical conditions without the inconvenience of traveling to a clinical center.

### BRIEF SUMMARY OF THE INVENTION

The present invention provides a system and associated method for use in remote patient management that includes monitoring the status of communication pathways during remote programming sessions to promote safe and complete transmission of programming data. The system includes a central programmer, which may be implemented as an Internet-based utility available on a web browser or on a computer at a clinic or other patient management center. The central programmer is linked to a remote programmer/monitor device via a communications network. The remote programmer/monitor is adapted for telemetric communication with an implantable medical device. The remote programmer/monitor receives programming data from the central programmer via the communications network and transfers the programming data to the IMD via telemetry. The system further includes a communication monitoring module for monitoring the status of the communication network link, the telemetry link, and the continuity of communication connectivity between system components. A display is provided for presenting a communication status determined from the monitored communication link and telemetry link signals.

The associated method includes monitoring the communication link and telemetry link signals during a remote telemetry session and determining a communication link status for display on the central programmer. Link signals that are monitored may include telemetry signal strength, communication link bandwidth, and the change in signal strength or bandwidth over time. Each of these parameters may be displayed individually in a link status display. Alternatively, one or more parameters may be combined in a link status index and displayed. The link status display may be a fluctuating bar, a numerical status value, a graphical display, or an iconic display indicating an acceptable link or an unacceptable link.

The associated method may further include performing a comparative analysis of a link signal to criteria defining an acceptable link signal, such as a predetermined threshold or range of values for the telemetry signal strength or the communication link bandwidth. If the link signal is determined to be unacceptable, i.e., the communication link is too slow or erratic according to the monitored communication link signals or the telemetry signal strength is too low or erratic, a fail-safe response may be executed by the system or system functionality may be reduced.

In one embodiment, the fail-safe response is to restore a set of programming data that is considered to be a safe. This safe programming data may be the most recent set of programmed parameters prior to the initiation of the current telemetry session or another set of predefined default parameters. Reduced system functionality may be appropriate in the event that the communication bandwidth across a communication network is limited but stable. One fail-safe response may be to limit or eliminate the communication and subsequent display of real-time data received from the IMD, such as real-time cardiac waveforms.

In yet another embodiment, the communication connectivity across system components, including the IMD, the telemetry system, the programmer system logic, the programmer presentation logic, and the display, is monitored. If a loss of communication occurs at any point across the system, a fail-safe response is provided.

### BRIEF DESCRIPTION OF THE DRAWINGS

FIG. 1 illustrates a general scheme by which remote patient management may be performed.

FIG. 2 is a block diagram of typical functional components of an IMD, such as the IMD shown in FIG. 1.

FIG. 3 is a functional block diagram of typical components included in a remote programmer used in programming an IMD, such as the remote programmer shown in FIG. 1.

FIG. 4 is a block diagram illustrating the points of communication across a system used for remote programming of an IMD that may be interrupted during a remote programming session.

FIG. 5 is a flow chart summarizing steps included in a method for monitoring and displaying communication status during remote programming of a medical device.

FIG. 6 is an illustration of one example of a communication status display.

### DETAILED DESCRIPTION

The invention pertains to remote programming of an implantable medical device (IMD). During a remote programming session, an adverse event, such as a communication failure or signal interference, may prevent the proper completion of programming or result in improper programming due to data corruption or other factors. To promote safe and secure remote programming, the invention provides communication status monitoring to ensure that a communication network link and a telemetry link existing between a centralized programmer and a remote IMD meets a minimum set of safety requirements during transfer of programming data. The invention provides for the display of the communication status to a user and provides a fail-safe response should the communication status not meet the safety requirements.

A fail-safe response may include notifying a person of the communication failure during the programming session, modifying programmed operating parameter values within the implantable medical device, or delivering a therapy to a patient. Additional details regarding provision of a fail-safe response during remote programming are provided in U.S. patent application Ser. No. (P11283), hereby incorporated herein by reference in its entirety.

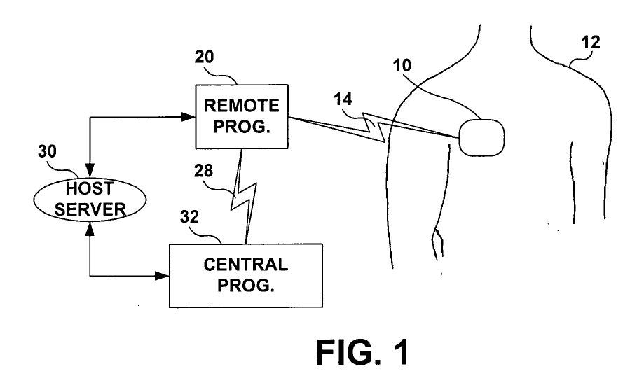

FIG. 1 illustrates a general scheme by which remote programming and monitoring of an IMD may be performed. The communication scheme shown enables continuous or periodic monitoring of patients in a hospital setting, in a home environment, or another location remote from a patient management center. The scheme further allows programming of a medical device in a remote location using a central patient management system. In this example, the medical device is shown as an implantable medical device (IMD) 10, implanted in a patient 12.

IMD 10 is provided with an antenna and associated circuitry, as will be described below, for establishing a bidirectional telemetry link 14 with a remote programmer 20, which may be embodied as a programmer or home monitor. Data acquired by IMD 10 can be transferred to remote programmer 20 through telemetry link 14. Remote programmer 20 is adapted to communicate with a central programmer 32 to allow transfer of data received from IMD 10 to the central programmer 32. Likewise, remote programmer 20 may receive programming data or commands from the central programmer 32 for transfer to IMD 10. Remote programmer 20 transfers data to and receives data from central programmer 32 via a communication link 28, which may be established via a local area network, a wide area network, a telecommunications network or other appropriate communications network and may be a wireless communication link.

Transfer of data between remote programmer 20 and central programmer 32 may occur via a networked host server 30. Other examples of communication schemes that may be used in remote monitoring systems are generally disclosed in U.S. Pat. No. 6,599,250 issued to Webb et al., U.S. Pat. No. 6,442,433 issued to Linberg, and U.S. Pat. No. 6,574,511 issued to Lee, U.S. Pat. No. 6,480,745 issued to Nelson et al., U.S. Pat. No. 6,418,346 issued to Nelson et al., and U.S. Pat. No. 6,250,309 issued to Krichen et al., all of which patents are incorporated herein by reference in their entirety.

Central programmer 32 includes a database for storing medical records and programs or algorithms for analyzing and presenting medical data on a display for viewing by a clinician. Central programmer 32 may further include a processor for converting data to or from a device format. Alternatively, data conversion may be performed by host server 30. To facilitate programming operations, a graphical user interface is used for viewing and selecting programming options and commands to be transferred to IMD 10. Central programmer 32 may be implemented on a computer located at a clinic or implemented on the Internet (via a host server 30), accessible using a web browser.

Implementation of the present invention is not limited to remote programming of implantable medical device systems as shown in FIG. 1. In other embodiments, the remote medical device associated with a patient that may be programmed from central programmer 32 may be an external medical device deployed for bedside or ambulatory monitoring or therapy delivery. The external medical device would be adapted for communication with the central patient management system via a communications network as generally shown in FIG. 1.

Central programmer 32 relies on an array of intermediate networking equipment along communication link 28 to communicate with remote programmer 20. The intermediate networking equipment, such as routers, servers, hubs, and the like, as well as software, such as web browsers, web servers, drivers, and other communication and rendering modules, may create potential faults, outages and other adverse events within communication link 28.

In general, the conglomeration of equipment and software along the communication link 28 creates an unpredictable, hazardous environment that presents reliability issues in the remote programming context. Transmission failures and data corruption are two possible effects of a hazardous environment. However, the sensitive nature of IMDs requires stability of a controlled operating environment to promote viability of remote programming.

Remote programming is further complicated by telemetry between remote programmer 20 and IMD 10. As a result, a remote programming session may encounter an adverse event due to not only a failure in the communication link 28 between central programmer 32 and remote programmer 20, but also a failure in the telemetry link 14 between remote programmer 20 and IMD 10, e.g., due to interference or out-of-range conditions. In either case, a programming session failure or inappropriate IMD programming may occur.

A programming session failure includes a disruption in telemetry between IMD 10 and remote programmer 20 located proximate the implantable medical device, a disruption in communication link 28 between remote programmer 20 and central programmer 32, a loss of connection between clinician instrumentation and central programmer 32, or the like, and results in incomplete or improper programming. For example, a connection may be lost between two devices if a power interruption occurs. In addition, a connection may be lost if a device is out of range, or if the device suffers interference.

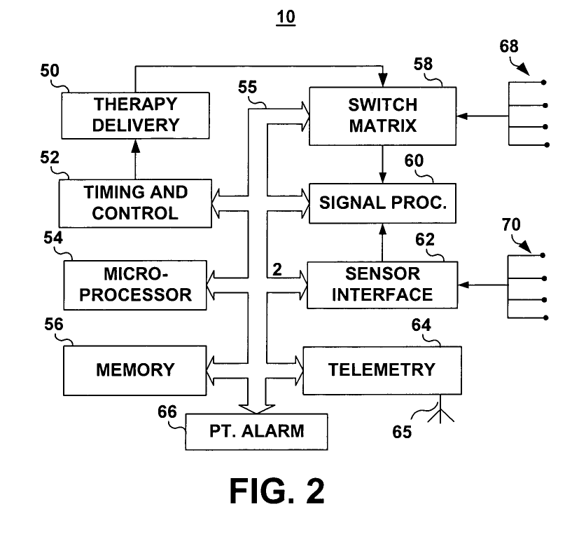

FIG. 2 is a block diagram of typical functional components of an IMD, such as the IMD shown in FIG. 1. IMD 10 generally includes timing and control circuitry 52 and a control system that may employ microprocessor 54 with associated firmware or a digital state machine for timing and controlling sensing and therapy delivery functions in accordance with a programmed operating mode. Microprocessor 54 and associated memory 56 are coupled to the various components of IMD 10 via a data/address bus 55. IMD 10 may include therapy delivery unit 50 for delivering a therapy, such as an electrical stimulation or drug therapy, under the control of timing and control unit 52. In the case of electrical stimulation therapies, such as cardiac stimulation therapies, therapy delivery unit 50 is typically coupled to two or more electrodes 68 via a switch matrix 58. Switch matrix 58 is used for selecting which electrodes and corresponding polarities are used for delivering electrical stimulation pulses.

Electrodes used for sensing and electrodes used for stimulation may be selected via switch matrix 58. When used for sensing, electrodes 68 are coupled to signal processing circuitry 60 via switch matrix 58. Signal processor 60 includes sense amplifiers and may include other signal conditioning circuitry and an analog-to-digital converter. Electrodes 68 may also be used for sensing electrical signals within the body, such as cardiac signals, or for measuring impedance. In the case of cardiac stimulation devices, cardiac electrical signals are sensed for determining when an electrical stimulation therapy is needed and in controlling the timing of stimulation pulses. In other embodiments, electrodes 68 may be used for measuring impedance signals for monitoring, for example, edema, respiration or heart chamber volume.

IMD 10 may additionally or alternatively be coupled to one or more physiological sensors 70. Such sensors may include pressure sensors, accelerometers, flow sensors, blood chemistry sensors, activity sensors or other physiological sensors known for use with IMDs. Sensors 70 are coupled to IMD 10 via a sensor interface 62 which provides sensor signals to signal processing circuitry 60. Sensor signals are used by microprocessor 54 for detecting physiological events or conditions. For example, IMD 10 may monitor heart wall motion, blood pressure, blood chemistry, respiration, or patient activity. Monitored signals may be used for sensing the need for delivering a therapy under control of the control system. Physiological data may be recorded continuously by IMD 10 or upon a detected triggering event or change in a monitored physiological condition. Acquired physiological data may be stored for later transfer to an external programmer or monitor or transferred in real-time.

The control system includes associated memory 56 for storing a variety of programmed-in operating mode and parameter values that are used by microprocessor 54. The memory 56 may also be used for storing data compiled from sensed physiological signals and/or relating to device operating history for telemetry out on receipt of a retrieval or interrogation instruction. All of these functions and operations are known in the art, and many are generally employed to store operating commands and data for controlling device operation and for later retrieval to diagnose device function or patient condition. Microprocessor 54 controls device diagnostic functions, such as lead impedance monitoring, stimulation threshold testing, and device longevity estimation. Microprocessor 54 may also manage the storage of device performance parameters such as pacing capture success rate, frequency of delivered therapies, and response to delivered therapies. Device-related parameters acquired by IMD 10 may be transferred to an external programmer for evaluating device function.

Microprocessor 54 may be programmed to generate an alert or alarm notifications in response to detecting predetermined physiological or device-related conditions or events. A patient alert signal is generated by patient alarm circuitry 66. A patient alert signal may be an audible sound or a perceivable vibration or other sensory stimulation. In one embodiment of the invention, a patient alert may be generated upon detection of a poor communication status or loss of communication in conjunction with the execution of a fail-safe response to a communication failure during a remote programming session.

IMD 10 is equipped with telemetry circuitry 64 and antenna 65 for bidirectional communication with an external programmer. Programming data and monitoring data are transmitted during downlink or uplink telemetry between IMD telemetry circuitry and external telemetry circuitry included in the programmer. In an exemplary embodiment, telemetry circuitry 64 and antenna 65 are implemented as a long range telemetry system which allows IMD-to-programmer communication to occur without user intervention, e.g. without the use of a programming head. Communication between IMD 10 and an external programmer may occur while the patient moves freely within the telemetry range of the external telemetry circuitry.

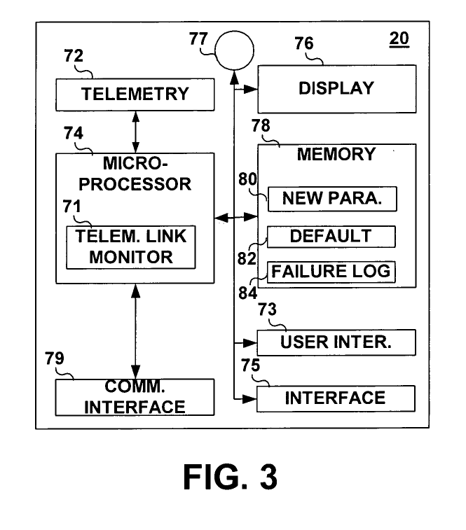

FIG. 3 is a functional block diagram of typical components included in a remote programmer used in programming an IMD, such as remote programmer 20 shown in FIG. 1. Remote programmer 20 is located at a remote location, such as a patient's home or in a clinic or other medical facility away from the central patient management system. Remote programmer 20 includes a communication network interface 79, such as a modem, used for communicating with central programmer 32. Remote programmer 20 acts as communication conduit between the central programmer 32 and IMD 10. Remote programmer 20 transfers programming data received from the central programmer 32 to the IMD 10. Remote programmer 20 also transfers data retrieved from the IMD 10 to the central programmer 32. During such transfers, a communication status is determined and displayed by the central programmer 32 and optionally on remote programmer 20. Aspects of the present invention for displaying a communication status may be incorporated in any system used for remotely programming an IMD or other external medical device, such as systems including a home monitor, patient programmer, or other remote device enabled for programming or interrogating an IMD.

Remote programmer 20 shown in FIG. 3 includes a telemetry circuit 72 for bidirectional communication with IMD 10. Remote programmer 20 includes a microprocessor 74 operating with associated memory 78 for controlling programmer functions.

Memory 78 may be used alone or in combination with IMD memory 56 (FIG. 2) to store a set of fail-safe programming data. Fail-safe programming data may be implemented by the IMD when a communication failure is detected. A telemetry link monitoring module is included in microprocessor 74 for monitoring uplink and downlink telemetry signal strength during a remote programming session. The telemetry link monitor 71 provides a signal for transfer to the central programmer 32 for presentation on a display such that a clinician may view the status of the telemetry link during a remote programming session. A programming unit capable of generating a telemetry status feedback signal is generally disclosed in U.S. Pat. No. 5,324,315, issued to Grevious, hereby incorporated herein by reference in its entirety.

Memory 78 may provide temporary storage of new, remotely programmed operating parameter values 80 and a set of default operating parameter values 82 used for restoring operating parameters during a fail-safe response. New operating parameter values may be stored temporarily during a remote programming session until communication safety criteria are satisfied and the new parameter values can be safely implemented by IMD 10. If a communication failure is detected, remote programmer 20 may maintain a failure log 84 for storing information relating to the communication failure. Such information can be useful in preventing or avoiding communication failures in the future.

In order for a clinician, patient, or caregiver to interact with remote programmer 20, a keyboard, pointing device, or other user interface 73, coupled to microprocessor 74, is provided. Display 76, which may be embodied as a graphical user interface, and/or the user interface 73 allow a user to enter command signals to initiate transmissions of downlink or uplink telemetry and to initiate and control telemetry sessions once a telemetry link with an implanted device has been established. Other types of user interaction mechanisms and electronics may be implemented such as voice recognition/response systems.

Display 76 may be included to display patient related data, menu choices and data entry fields used for entering data during a telemetry session. Display 76 can display a variety of screens of retrieved IMD data, previously stored or in real time and may display uplinked event signals as they are received and thereby serve as a means for enabling the user to reviewing IMD operating history and status. Display 76 may be used for displaying messages to a user regarding communication and telemetry link status during the communication with a central programmer 32 and IMD 10. A speaker 77 may also be provided for broadcasting audible tones or messages used to alert the user to communication link, telemetry, programming, and/or interrogation status or conditions. Remote programmer 20 may further include an interface 75 for coupling peripheral devices which may include external monitoring equipment such as ECG leads, blood pressure monitor, etc.

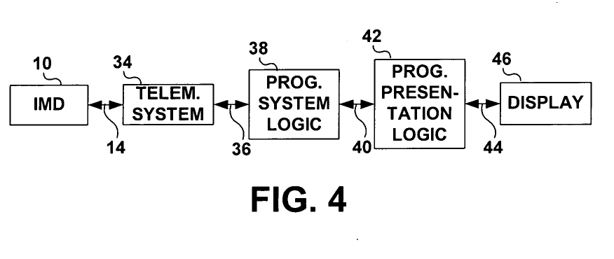

FIG. 4 is a block diagram illustrating the points of communication across a system used for remote programming of an IMD that may be interrupted during a remote programming session. The block diagram shown in FIG. 4 represents the logical partitioning of software utilized by the hardware system shown in FIG. 1. The software used for controlling and managing remote programming operations is designed to operate in a distributed environment. As such, the software, including software for monitoring communication status during remote programming sessions, may be implemented in several possible configurations

In one embodiment, telemetry system 34 is implemented in remote programmer 20 (shown in FIG. 1) providing communication between programmer system logic 38 and IMD 10. The programmer system logic 38, along with programmer presentation logic 42 and display 46 are implemented in central programmer 32 (FIG. 1). In an alternative embodiment, programmer system logic 38 is implemented on host server 30 (FIG. 1) and the presentation logic 42 and display 46 are implemented in central programmer 32. In yet another embodiment, programmer system logic 38 and the programmer presentation logic 42 are implemented in host server 30 and display 46 is implemented in central programmer 32. In still another embodiment, telemetry system 34 and programmer system logic 38 are implemented in remote programmer 20 and the programmer presentation logic may be implemented in either host server 30 or central programmer 32 with display 46 implemented in central programmer 32. In another embodiment, telemetry system 34, programmer system logic 38 and programmer presentation logic 42 may all be implemented in remote programmer 20 with display 46 implemented in central programmer 30. Thus, the distributed software for managing communication across the remote programming system may be implemented in a variety of configurations. In any of these configurations, communication links between the various components are subject to failure.

Telemetry link 14 existing between IMD 10 and telemetry system 34 is subject to interference or out-of-range errors. Telemetry system 34 includes any communication circuitry provided between IMD 10 and programming system logic 38. In one remote programming scenario, telemetry system 34 would include remote programmer 20 which acts as a communication conduit between IMD 10 and central programmer 32.

During remote programming sessions, a set of temporary programmed operation parameters may be implemented by IMD 10 for performing a diagnostic test. For example, a test may be performed on a patient having a pacemaker in which pacing therapies are temporarily disabled in order to obtain a recording of the patient's intrinsic heart rhythm. During temporary testing modes, a “retrigger” signal transferred from programmer system logic 38 via telemetry system 34 is utilized to maintain temporary programmed parameter values. If the. retrigger signal is not received by IMD 10, IMD 10 will initiate a fail-safe response, such as automatically reverting to permanently programmed values. Temporary or test parameters will be lost if the telemetry link 14 between IMD 10 and the telemetry system 34 is interrupted.

A logic control link 36 existing between telemetry system 34 and programmer system logic 38 is also subject to interruption or interference. Programmer system logic 38, which may be included in the central programmer 32 and/or host server 30 is provided for controlling remote programming operations. Programmer system logic 38 generates the retrigger signal to be transferred to IMD 10 via telemetry system 34 to cause IMD 10 to maintain temporary programmed values. If logic control link 36 between programmer system logic 38 and telemetry system 34 is interrupted, the retrigger signal cannot be transmitted and IMD 10 will revert to permanently programmed values.

A presentation control link 40 existing between programmer system logic 38 and programmer presentation logic 42 provides a bidirectional pathway for transferring data between programmer system logic 38 and display 46. Data received from programmer system logic 38 is formatted for viewing on display 46 by programmer presentation logic 42. Information displayed may be warnings or other messages generated by programmer presentation logic 42 in response to data received from programmer system logic 38. Other displayed information includes data received from IMD 10.

In accordance with the present invention, a communication status indicator is displayed by display 46 in response to monitoring communication status across telemetry link 14 and the overall communication link across the remote programming system which includes logic control link 36, presentation control link 40, and display link 44. To facilitate a successful programming session, communication between programmer system logic 38 and display 46 is required to present programming data, communication data, and patient data.

During a remote programming session the clinician interacts with display 46. The programmer system logic 38 will provide the programmer presentation logic 42 with notifications including request status and real-time data updates for presentation on display 46. If the presentation control link 40 is interrupted, programmer system logic 38 will issue a termination notification to telemetry system 34 to cause a fail-safe mechanism to occur, such as causing temporary programmed values to be aborted and permanent programmed values to be restored.

The programmer presentation logic 42 periodically polls programmer system logic 38. If programmer system logic 38 does not receive a poll from programmer presentation logic 42 for a predetermined period of time, programmer system logic 38 determines that communication has been lost at presentation control link 40 and will transmit a signal to telemetry system 34 to cause a fail-safe mechanism to be performed. Programmer presentation logic 42 may be receiving information from programmer system logic 38 but unable to send information requests to programmer system logic 38. In this scenario, programmer system logic 38 may send notification to programmer presentation logic 42 that the fail-safe mechanism has been performed such that this information may be presented on display 46.

Display 46 responds to user input by sending commands to programmer presentation logic 42 via communication link 44. Display 46 can also periodically request information from programmer presentation logic 42. If display 46 cannot communicate with programmer presentation logic 42 due to communication loss at display link 44, the programmer presentation logic 42 will not request updates from the programmer system logic 38. The programmer system logic 38 will determine loss of communication with display 46 in the absence of receiving an update request and transmit a fail-safe mechanism signal to telemetry system 34. Display 46 may generate a communication failure notification.

If the programmer presentation logic 42 is unable to communicate with display 46 to provide it with notifications, the programmer presentation logic 42 will return an error notification to programmer system logic 38. In response to the error notification, programmer system logic 38 will initiate a fail-safe mechanism.

Thus, the invention provides for a fail-safe mechanism to occur in response to a breach of communication at any point between the IMD 10 and display 46. The invention further provides for presentation of the communication status between display 46 and IMD 10 on display 46.

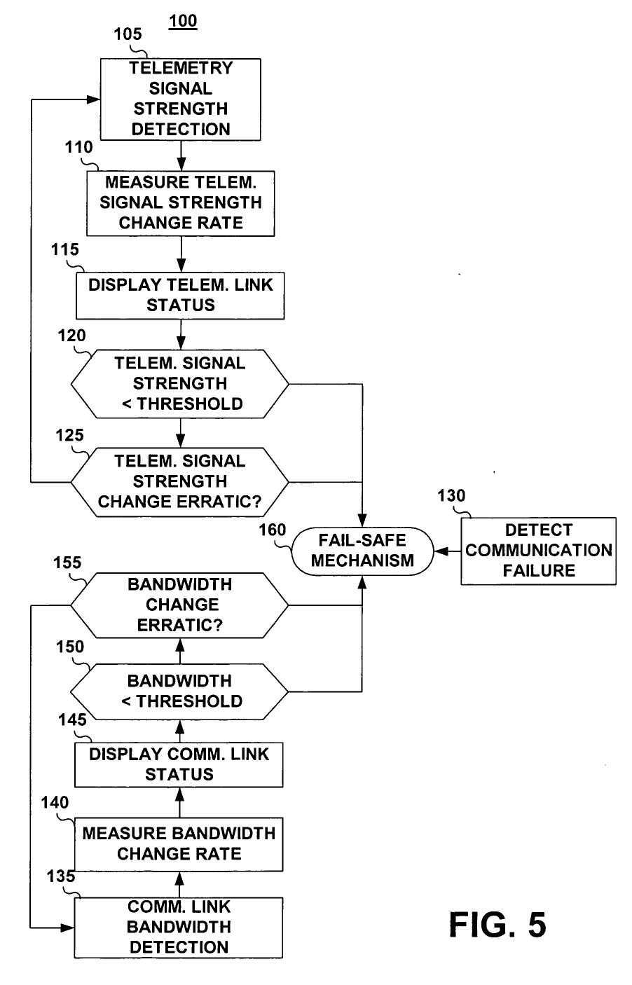

FIG. 5 is a flow chart summarizing steps included in a method for monitoring and displaying communication status during remote programming of a medical device. During a remote programming session, successful communication of data between an IMD and a central programmer occurs when communication across the various components shown in FIG. 4 remains intact. Such communication includes communication between system components across a distributed software environment as described above as well as communication through telemetry link 14 between IMD 10 and the remote programmer 20 and communication network link 28 between the remote programmer 20 and central programmer 32.

In method 100, a telemetry signal strength indicator is monitored by remote programmer 20 at step 105, which indicates the strength of the telemetry signal between remote programmer 20 and IMD 10, and may include both uplink and downlink signal strength. At step 110, the stability of the telemetry signal strength may be monitored by measuring the change in signal strength over time. By monitoring the change in signal strength over time, detection of an erratic telemetry signal may be made. At step 115, a telemetry link status signal is communicated to the programmer presentation logic 42 for presentation by display 46. A telemetry link status display may be updated at a programmable frequency, e.g. 30 to 600 Hz depending on the system capabilities. A clinician using the central programmer 32 may then observe the status of the telemetry link 14 between IMD 10 and remote programmer 20 (shown in FIG. 1).

At decision step 120, the programmer system logic 38 compares the signal strength indicator to a threshold telemetry link signal strength considered to be the minimal signal strength reliable for safe data transmission. If the signal strength is less than the threshold strength, a fail-safe mechanism is invoked at step 160.

At decision step 125, the rate of change in the received telemetry signal strength indicator is compared to a threshold rate of change to determine if the telemetry signal strength is erratic. If the signal strength is changing rapidly, data transfer may be unreliable. If an erratic telemetry signal strength is detected at decision step 125 based on an analysis of the signal strength rate change, a fail safe mechanism is invoked at step 160.

During a remote programming session, the communication link between a remote programmer/monitor and central programmer is also monitored as indicated at step 135. The monitored communication link includes communication links between the distributed control software such as the logic control link 36, presentation control link 40, and display link 44 as shown in FIG. 4. The communication link bandwidth is detected at step 135 and the rate of change of the communication link bandwidth is determined at step 140. This information is communicated to programmer presentation logic 42 (FIG. 4) for presentation on display 46 at step 145. The clinician interacting with the central programmer may then observe the communication link status, which may be displayed as a link speed and/or link stability, during the programming session.

The communication link bandwidth is compared to a bandwidth threshold at decision step 150. The bandwidth threshold is a predetermined value considered to be associated with the slowest communication link speed that allows reliable data transmission between the remote programmer and the central programmer. If the bandwidth is detected below the bandwidth threshold at decision step 150, a fail-safe mechanism is invoked at step 160.

The fail-safe mechanism may be made according to the communication speed detected. For example, if the communication link is fast enough for reliable data transmission but slower than necessary to communicate real-time data available from the IMD, the fail-safe response may be to censor or eliminate the real-time data transferred. Real-time data updates transferred to the central programmer may be reduced or some data eliminated (such as cardiac waveform data) to allow safe but reduced end-user remote programming functionality. As such, in some embodiments, a fail-safe response may be to reduce remote programming operation functionality rather than eliminate remote programming functionality when the communication status is determined acceptable for limited but stable functionality.

At decision step 155, the rate of bandwidth change is compared to an erratic link threshold. If the bandwidth is changing rapidly, the communication link may be too erratic to ensure reliable data transfer. A fail-safe mechanism is invoked at step 160 in response to the detection of an erratic communication link as determined at decision step 155. The response to an erratic telemetry link or erratic communication network link may be programmably enabled or disabled by a clinician. The communication safety thresholds set for a required telemetry link signal strength and a required communication network link speed may also be programmable.

A fail-safe mechanism may also be invoked at step 160 in response to a loss of communication detected at step 130 at any other point across the remote programming system, such as between programmer system logic 38, programmer presentation logic 42, and display 46 as described above in conjunction with FIG. 4.

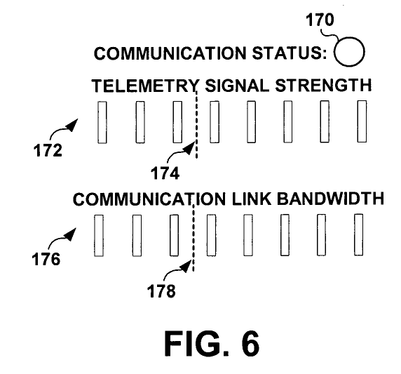

FIG. 6 is an illustration of one example of a communication status display. An overall communication status indicator 170 may be displayed. The overall communication status indicator 170 indicates that communication is intact across the system, for example by displaying a green light, or that a communication failure has occurred, for example by displaying a red light. The point of that the communication failure was detected may be displayed in an accompanying notation.

In addition or alternatively, a telemetry signal strength indicator 172 may be displayed, for example as a fluctuating bar moving left to right as changes in signal strength are updated, as shown in FIG. 6. The signal strength threshold 174 may be indicated such that the clinician is aware when the telemetry link signal strength is less than the threshold for safe telemetric communication.

Likewise, a communication link bandwidth indicator 176 may be displayed. Communication link bandwidth indicator 176 is also shown as a fluctuating bar indicator that moves left to right as changes in the communication link bandwidth are updated. The threshold bandwidth 178 for safe communication may be indicated.

Additionally, erratic signal indicators may be displayed for both the telemetry link and the communication network link. The remote programming system communication status may be displayed in a variety of numerical, graphical, or iconic formats indicating to the clinician the communication status across remote programming system components in an unambiguous and intuitive manner.

What is claimed is:

1. A system, comprising:
a remote programmer adapted for telemetric communication with an implantable medical device for use in proximity to a patient having the implantable medical device;
a communication network coupled to the remote programmer;
a central programmer coupled to the communication network for transferring programming commands to the implantable medical device via the remote programmer; and
a communication status module for monitoring the communication between the central programmer and the implantable medical device and for generating a communication status signal.

2. The system according to claim 1 wherein the communication status module comprises a communication network link bandwidth monitor for monitoring a communication link bandwidth on the communication network and wherein the communication status signal comprises a bandwidth status signal.

3. The system according to claim 1 wherein the communication status module comprises a telemetry link signal strength monitor for monitoring a telemetry link signal strength between the implantable medical device and the remote programmer and wherein the communication status signal comprises a telemetry strength status signal.

4. The system according to claim 1 wherein the communication status module comprises a communication link bandwidth change monitor for detecting an erratic communication link bandwidth and wherein the communication status signal comprises an erratic bandwidth status signal.

5. The system according to claim 1 wherein the communication status module comprises a telemetry link signal strength change monitor for detecting an erratic telemetry link signal strength and wherein the communication status signal comprises an erratic telemetry strength signal.

6. The system according to claim 1 further comprising a display for receiving the communication link status signal and displaying a communication status.

7. A system, comprising:
means for generating a set of programming data for use in programming an implantable medical device;
means for transmitting the programming data via a communication network;
means for receiving the programming data from the communication network;
means for monitoring signals on the communication network during transmission of programming data from the generating means to the receiving means and for generating a communication status indicator; and
means for displaying the communication status indicator.

8. The system according to claim 7 further comprising means for performing a fail-safe response responsive to the communication status indicator.

9. The system according to claim 8 wherein the means for performing a fail-safe response comprises means for restoring permanently programmed operating parameter values in the implantable medical device.

10. The system according to claim 8 wherein the means for performing a fail-safe response comprises means for eliminating at least a portion of real-time data for transfer from the implantable medical device.

11. A method for use during remote programming of a medical device, comprising:
monitoring a communication signal during transmission of programming data between a central programmer and the remote medical device; and
displaying a communication status responsive to the monitored communication signal.

12. The method according to claim 11 wherein the monitored communication signal comprises a communication link bandwidth on a communication network.

13. The method according to claim 11 wherein the monitored communication signal comprises a rate of change in the communication link bandwidth over time.

14. The method according to claim 11 wherein the monitored communication signal comprises a telemetry signal strength.

15. The method according to claim 11 wherein the monitored communication signal comprises a rate of change in the telemetry signal strength over time.

16. The method according to claim 11, further comprising providing a fail-safe response responsive to the monitored communication signal.

17. The method according to claim 16 wherein providing a fail-safe response comprises restoring a previously programmed operating parameter value in the medical device.

18. The method according to claim 16 wherein providing a fail-safe response comprises eliminating at least a portion of real-time data for transfer from the implantable medical device to the central programmer.
